(19) United States
(12) Patent Application Publication
(10) Pub. No.: US 2024/0404632 A1
(43) Pub. Date: Dec. 5, 2024

(54) Method for Sequence-Based Prediction of Controlled Terms and Generating Protein Sequences from Controlled Terms using Enhanced Large Language Models

(71) Applicant: Sarah Wenhsia Ho, Los Altos Hills, CA (US)

(72) Inventor: Sarah Wenhsia Ho, Los Altos Hills, CA (US)

(21) Appl. No.: 18/227,977

(22) Filed: Jul. 31, 2023

Related U.S. Application Data
(60) Provisional application No. 63/470,159, filed on May 31, 2023.

(51) Int. Cl. G16B 20/30; G06N 3/0455; G06N 3/0475; G06N 3/0895; G06N 3/09; G16B 30/00; G16B 40/20

(52) U.S. Cl. CPC G16B 20/30; G06N 3/0455; G06N 3/0475; G06N 3/0895; G06N 3/09; G16B 30/00; G16B 40/20

(57) ABSTRACT

The present invention relates to a method for enhancing the creativity of a generative pre-trained Large Language Model (LLM) in protein sequence generation and predicting controlled terms from protein sequences. The method includes incorporating 22 novel names representing the 22 amino acids into the vocabulary of the pre-trained LLM, conducting self-supervised learning using protein sequences encoded with the novel names to improve the LLM's comprehension and generation of coherent protein sequences, performing supervised learning using protein sequences to refine the LLM's ability to predict controlled terms based on protein sequences, and performing supervised learning to refine the LLM's ability to generate protein sequences based on controlled terms. The method includes generating the novel names either through a computer program or manually and utilizing datasets of protein sequences and their corresponding controlled terms from a protein database. The self-supervised learning employed is a masked language model (MLM). Additionally, an alternative method is disclosed, which involves identifying a set of 22 amino acid names from the original vocabulary of the pre-trained LLM and proceeding with self-supervised and supervised learning steps using the selected names. The two methods can be used independently or in combination to enhance the creativity of the LLM in achieving protein sequence generation and predicting controlled terms from protein sequences.

### CROSS-REFERENCE TO RELATED APPLICATIONS

This application claims the benefit of priority under 35 U.S.C. § 119 (e) to the following:
Provisional Patent Application No. 63/470,159, filed on 31 May 2023.

The disclosures of the above application are incorporated herein by reference in their entirety.

### FEDERALLY SPONSORED RESEARCH AND DEVELOPMENT

No part of the claimed subject matter was made with government support.

### JOINT RESEARCH AGREEMENT

N/A.

### REFERENCE TO A “SEQUENCE LISTING”, A TABLE, OR A COMPUTER PROGRAM LISTING APPENDIX SUBMITTED ON A COMPACT DISC AND AN INCORPORATION-BY-REFERENCE OF THE MATERIAL ON THE COMPACT DISC

N/A.

### PRIOR ART

### Citations

1) Vaswani, A., et al. “Attention is all you need.” In: Advances in Neural Information Processing Systems 30. 2017. pp. 5998-6008.
2) Elnaggar, A., et al. “ProtTrans: Towards Cracking the Language of Life's Code Through Self-Supervised Deep Learning and High Performance Computing.” bioRxiv. 2020.
3) Madani, A., et al. “Deep neural language modeling enables functional protein generation across families.” bioRxiv. 2021.
4) Madani, A., et al. “Large language models generate functional protein sequences across diverse families.” Nature Biotechnology. 2023.
5) Ni, B., et al. “Generative design of de novo proteins based on secondary-structure constraints using an attention-based diffusion model.” Chem. 2023.
6) US Patent Reference 20230123770. “Protein database search using learned representations.” Publication Date: Apr. 20, 2023

Proteins are essential for life and perform a wide variety of functions in cells, including structural support, catalyze chemical reactions, and transmit signals. The sequence-structure-function relationship is the central problem of protein biology. It is the study of how the sequence of amino acids in a protein determines its structure and function, and it is essential for understanding disease mechanisms and developing proteins and pharmaceuticals for use in medical treatment.

Computational methods are used to predict various properties of proteins, including their structure, function, interactions, and dynamics. These methods use protein sequence as input and can provide valuable insights into the behavior of proteins. By analyzing the sequence, computational tools can predict structural features, such as secondary and tertiary structures, as well as functional characteristics, such as enzyme activity and ligand binding sites.

There are two approaches that use computational methods to predict protein functions: 1. Sequence-based methods: These methods predict protein function based on the sequence of amino acids that make up the protein. Examples include homology-based methods, which compare the sequence of the protein in question to sequences of known proteins with similar functions, and machine learning-based methods that use sequence features to predict function. 2. Structural methods: These methods predict protein function based on the 3D structure of the protein. Examples include structure-based function prediction, which compares the protein's structure to structures of known proteins with similar functions, and ligand-binding assays, which test whether the protein binds to specific molecules.

One can predict protein functions from protein sequences through either Sequence-based methods or Structural methods. Sequence-based methods directly analyze the sequence of amino acids that make up the protein, while Structural methods first predict the protein's 3D structure and then use it to infer functions.

There are many ways to predict protein structure. Some of the most common computational methods include first-principles-based structural simulations (ab initio methods), molecular dynamics simulations and homology modeling. Machine learning approaches, such as artificial neural networks, protein threading, and fold recognition, have also been widely used in protein structure prediction. Among these, the transformer architecture of neural networks, which uses a self-attention mechanism to process input sequences, has shown particular promise in achieving high accuracy in prediction tasks.

Several related tools use the methods described above, including GROMACS, SWISS-MODEL, I-TASSER, Phyre2, RaptorX, and BLAST. While these tools have been successful in certain contexts, they all suffer from various limitations, such as limited accuracy, scalability, and generalizability. AlphaFold2 is a new protein structure prediction tool developed by DeepMind that overcomes many of these limitations. AlphaFold2 uses a deep learning approach that is able to achieve high accuracy, scalability, and generalizability. In the CASP14 experiment, AlphaFold2 was the top-performing method, outperforming all other methods by a significant margin. It is the first to use transformers for protein structure prediction. The transformer is able to learn long-range dependencies in the protein sequence, which is essential for accurately predicting the protein structure.

Turning to Sequence-based methods, they rely on analyzing the sequence of amino acids that make up a protein to predict its function. There are several different types of sequence-based methods that can be used, including homology-based methods, hidden Markov models, and machine learning-based methods. Homology-based methods compare the sequence of the protein in question to sequences of known proteins with similar functions. Hidden Markov models identify patterns in the sequence that are indicative of certain functions. Machine learning-based methods have become increasingly popular in recent years. These methods use advanced algorithms, such as convolutional neural networks (CNNs), to analyze protein sequences and predict their functions. There are now many existing tools that use CNNs to predict protein functions from protein sequences, including DeepGO, DeepBind, DeepDTA, and DeepFam. More advanced tools, such as DeepLoc 2.0, use protein language models to make even more accurate predictions.

The most advanced protein language models also use the Transformer architecture. Transformer was first introduced in natural language processing (NLP) in a 2017 paper by Vaswani et al. and has since been adapted to learn and understand protein sequences. These models learn meaningful representations of proteins (protein-LM embeddings) in a self-supervised manner by using the vast amount of unlabeled sequences contained in protein databases such as UnitProt, Swiss-Prot, Pfam, UniRef, and metagenomic databases such as the big fantastic database (BFD). The first protein language model using the Transformer architecture was first introduced in ProtTrans, a 2020 paper by Ahmed Elnaggar et al. The protein-LM embeddings, derived from Transformer models trained on protein sequences, have demonstrated potential in predicting various protein functions, including subcellular localization (DeepLoc 2.0), and phenotype values (as described in US Patent Reference 20230123770).

After elucidating the computational methods utilized for predicting various properties of proteins, our focus now shifts to the realm of computational protein design. This innovative field, situated at the crossroads of bioinformatics, computer science, and molecular biology, centers on the intentional and computational manipulation of protein structures and sequences, culminating in the creation of novel proteins endowed with specific functionalities. Since the groundbreaking success of de novo-designed protein Top7 in 2003, computational protein design has continually evolved, giving rise to novel strategies that advance the creation of proteins with desired functions.

One such notable advancement comes from the realm of generative design, where a 2023 paper by Bo Ni et al. introduces the use of an attention-based diffusion model to generate de novo proteins based on secondary-structure constraints. Additionally, a recent development involves the integration of protein language models and transformer architectures. In a 2023 paper by Ali Madani et al., large language models demonstrate their prowess in generating functional protein sequences across diverse families, revolutionizing the landscape of computational protein design.

### BACKGROUND OF THE INVENTION

In recent years, there has been a notable trend of applying protein sequences to transformer-based language models for predicting protein functions and facilitating protein design across numerous patents, projects, and academic papers since 2020. These language models have been specifically trained on the protein space, taking protein sequences as input and generating either protein sequences or learned embeddings in the form of vector representations.

However, it has been observed that these protein language models have not fully harnessed the immense capabilities of large language models (LLM). LLMs are trained on massive datasets of text, and they can learn to represent the meaning of words and phrases in a very sophisticated way. This makes them powerful tools for natural language processing tasks, such as machine translation, text summarization, and question answering.

It has been challenging to train LLMs on protein space and to leverage their capabilities in the context of protein function predictions and protein design, we propose a method that starts with a generative LLM that has been pre-trained on a large dataset of natural language text. We then fine-tune the model on a dataset of protein sequences. This allows the model to learn the relationships between words and phrases in the natural language space and the corresponding amino acids in protein sequences. Our method can be used on a variety of protein-related tasks, such as protein function prediction and protein design.

We believe that our invention has the potential to revolutionize the field of protein engineering and design. By enabling the use of natural language queries to instruct LLMs to generate proteins with specific desired functions, we can create new possibilities for the development of new drugs, enzymes, and other proteins with valuable properties.

### SUMMARY OF THE INVENTION

This method involves enhancing a generative pre-trained Large Language Model (LLM) with 22 new names, each representing a unique amino acid. The process employs a combination of self-supervised and supervised learning.

The pre-trained LLMs, originally trained on extensive datasets of natural language text, possess the capability to comprehend and represent the meaning of words and phrases effectively.

The method begins by creating unique novel names, which are not a part of the pre-trained LLM's vocabulary. The LLM then undergoes self-supervised learning, using all protein sequences available in protein databases represented by these newly introduced names. This training enables the LLM to understand and generate coherent sequences involving these names.

Next, the method employs supervised learning, using protein sequences as inputs and their associated controlled terms as outputs. This fine-tuning process enables the LLM to predict controlled terms based on any given protein sequences accurately.

To further enhance the LLM's capabilities, the model is subjected to additional supervised learning steps. Here, the LLM is trained using a dataset where the controlled terms act as inputs and the corresponding novel names are the outputs. Through this process, the LLM learns to generate appropriate novel name sequences when provided with controlled terms. As a result, the LLM can generate coherent and meaningful protein sequences based on the provided controlled terms, paving the way for valuable and coherent protein design.

Alternatively, the method can start by choosing a set of 22 amino acid names already present in the pre-trained LLM's vocabulary. Table 1 in the DETAILED DESCRIPTION OF THE INVENTION section lists these selected names. Using these pre-existing names can help fine-tune the model by leveraging the LLM's pre-trained knowledge of these amino acids.

The LLM undergoes self-supervised learning once more, this time with protein sequences represented by these selected names. This phase enhances the LLM's understanding of the inherent patterns within the selected amino acid names. Next, supervised learning is applied, with protein sequences using the selected names as inputs and their corresponding controlled terms as outputs. This process reinforces the association between the protein sequences and the desired output within the LLM. Finally, additional supervised learning steps are introduced, where controlled terms are provided as inputs, and the desired name sequences (selected names) act as outputs. Through this process, the LLM learns to generate functional protein sequences represented by selected names when given input controlled terms.

In conclusion, this method effectively enhances a pre-trained Large Language Model (LLM) for advanced protein function prediction and design. By introducing novel or selected amino acid names into the model and utilizing a combination of self-supervised and supervised learning techniques, the LLM is trained to accurately predict controlled terms and generate coherent protein sequences. This represents a significant advancement in the field of bioinformatics, paving the way for more sophisticated and accurate protein design based on natural language processing techniques.

### BRIEF DESCRIPTION OF DRAWINGS

In FIG. 1, the process starts with accessing protein databases. A specific database, UniProt, is used in this implementation to gather a rich dataset of protein sequences. The dataset is then utilized to fine-tune the LLM through masked language modeling methodology. The objective is to train the LLM to generate coherent protein sequences.

During self-supervised learning, the protein sequences are represented as strings of amino acid symbols. Each symbol corresponds to a specific amino acid residue. In the training process, the input protein sequences derived from the protein databases are mapped to sequences of new names. This mapping involves replacing each amino acid symbol with a specific new name associated with it. The LLM is trained using this transformed dataset to develop a comprehensive understanding and proficiency in generating coherent protein sequences.

For example, the original protein sequence “NLYIQWLKDGGPSSGRPPPS” for Trp-Cage is mapped to a corresponding sequence where each amino acid symbol is replaced with a specific new name shown as follows:

Asnxaeiou-Leuxaeiou-Tyrxaeiou-Ilexaeiou-Glnxaeiou-Trpxaeiou-Leuxaeiou-Lysxaeiou-Aspxaeiou-Glyxaeiou-Glyxaeiou-Proxaeiou-Serxaeiou-Serxaeiou-Glyxaeiou-Argxaeiou-Proxaeiou-Proxaeiou-Proxaeiou-Serxaeiou

FIG. 2 describes supervised learning where the Large Language Model (LLM) is trained to map input data (protein sequence) to corresponding output data (controlled terms). Each row represents a pair of input and output data. The dataset consists of pairs of parallel texts, where one text is in the protein sequence represented by novel names, and the corresponding text is in the controlled terms.

During training, the LLM processes the input data and generates predictions for the corresponding output data. The model's parameters are adjusted through optimization techniques based on the comparison of the predicted data with the ground truth output data (controlled terms) from the dataset.

The loss function measures the discrepancy between the predicted data and the ground truth controlled terms from the output data.

Through iterative training over multiple examples, the LLM learns to capture the patterns and associations between the input and output data, enabling it to generate accurate controlled terms when provided with new protein sequences.

FIG. 3 describes supervised learning where the Large Language Model (LLM) is trained to map the input data (controlled terms) to the corresponding output data (protein sequence). Each row represents a pair of input and output data. The dataset consists of pairs of parallel texts, where one text is in the controlled terms, and the corresponding text is in the protein sequence represented by novel names.

During training, the LLM processes the input data and generates predictions for the corresponding output data. The model's parameters are adjusted through optimization techniques based on the comparison of the predicted data with the ground truth output data (protein sequence) from the dataset.

The loss function measures the discrepancy between the predicted data and the ground truth protein sequence from the output data.

Through iterative training over multiple examples, the LLM learns to capture the patterns and associations between the input and output data, enabling it to generate accurate protein sequences when provided with new controlled terms.

In FIG. 4, the process starts with accessing protein databases. A specific database, UniProt, is used in this implementation to gather a rich dataset of protein sequences. The dataset is then utilized to fine-tune the LLM through masked language modeling methodology. The objective is to train the LLM to generate coherent protein sequences.

During self-supervised learning, the protein sequences are represented as strings of amino acid symbols. Each symbol corresponds to a specific amino acid residue. In the training process, the input protein sequences derived from the protein databases are mapped to sequences of amino acid names. This mapping involves replacing each amino acid symbol with a specific amino acid name associated with it. The LLM is trained using this transformed dataset to develop a comprehensive understanding and proficiency in generating coherent protein sequences.

For example, the original protein sequence “NLYIQWLKDGGPSSGRPPPS” for Trp-Cage is mapped to a corresponding sequence where each amino acid symbol is replaced with a specific amino acid name shown as follows:

Asparagine-Leucine-Tyrosine-Isoleucine-Glutamine-Tryptophan-Leucine-Lysine-Aspartic-Glycine-Glycine-Proline-Serine-Serine-Glycine-Arginine-Proline-Proline-Proline-Serine

FIG. 5 describes supervised learning where the Large Language Model (LLM) is trained to map input data (protein sequence) to corresponding output data (controlled terms). Each row represents a pair of input and output data. The dataset consists of pairs of parallel texts, where one text is in the protein sequence represented by amino acid names, and the corresponding text is in the controlled terms.

During training, the LLM processes the input data and generates predictions for the corresponding output data. The model's parameters are adjusted through optimization techniques based on the comparison of the predicted data with the ground truth output data (controlled terms) from the dataset.

The loss function measures the discrepancy between the predicted data and the ground truth controlled terms from the output data.

Through iterative training over multiple examples, the LLM learns to capture the patterns and associations between the input and output data, enabling it to generate accurate controlled terms when provided with new protein sequences.

FIG. 6 describes supervised learning where the Large Language Model (LLM) is trained to map the input data (controlled terms) to the corresponding output data (protein sequence). Each row represents a pair of input and output data. The dataset consists of pairs of parallel texts, where one text is in the controlled terms, and the corresponding text is in the protein sequence represented by amino acid names.

During training, the LLM processes the input data and generates predictions for the corresponding output data. The model's parameters are adjusted through optimization techniques based on the comparison of the predicted data with the ground truth output data (protein sequence) from the dataset.

The loss function measures the discrepancy between the predicted data and the ground truth protein sequence from the output data.

Through iterative training over multiple examples, the LLM learns to capture the patterns and associations between the input and output data, enabling it to generate accurate protein sequences when provided with new controlled terms.

### DETAILED DESCRIPTION OF THE INVENTION

We have developed a novel model based on a generative pre-trained Large Language Model (LLM) to tackle the prediction of controlled terms from protein sequences and the generation of protein sequences from controlled terms. The purpose of this new model is to extend the LLM's abilities and make it suitable for handling these particular tasks. The LLM referred to here is a generative model known for its creativity in tasks like text generation.

The model we present follows a two-fold approach: predicting controlled terms from protein sequences and generating protein sequences from controlled terms. By incorporating both aspects, we create a comprehensive method that facilitates bidirectional interactions between protein sequences and controlled terms.

### Step 1: Generation of 22 Novel Names

The first step entails the generation of 22 novel names that are not currently part of the pre-trained LLM's vocabulary. We employed a specific large language model (LLM) known as Llama 2 for our implementation. However, it should be emphasized that the disclosed method can be applied to any other LLM that shares similar characteristics, including potential models such as GPT-J, GPT-NeoX, GPT-4. The described techniques and processes are not limited to a particular LLM and can be adapted for use with various LLM architectures or future advancements in language modeling technology. We have introduced 22 novel names in our study, each representing one of the 22 amino acids. To ensure their uniqueness, we carefully cross-referenced these novel names with the existing tokens in the pre-trained LLM's vocabulary. This ensured that the 22 novel names were distinct from the pre-existing tokens. For a comprehensive list of the 22 novel names and their corresponding amino acids, please refer to Table 1.

TABLE 1
Mapping of novel names to Amino Acids.

| Amino Acid Name | Novel Name | 3 Letter Code | 1 Letter Code |
|---|---|---|---|
| Alanine | Alaxaeiou | Ala | A |
| Arginine | Argxaeiou | Arg | R |
| Asparagine | Asnxaeiou | Asn | N |
| Aspartic | Aspxaeiou | Asp | D |
| Cysteine | Cysxaeiou | Cys | C |
| Glutamine | Glnxaeiou | Gln | Q |
| Glutamic | Gluxaeiou | Glu | E |
| Glycine | Glyxaeiou | Gly | G |
| Histidine | Hisxaeiou | His | H |
| Isoleucine | Ilexaeiou | Ile | I |
| Leucine | Leuxaeiou | Leu | L |
| Lysine | Lysxaeiou | Lys | K |
| Methionine | Metxaeiou | Met | M |
| Phenylalanine | Phexaeiou | Phe | F |
| Proline | Proxaeiou | Pro | P |
| Serine | Serxaeiou | Ser | S |
| Threonine | Thrxaeiou | Thr | T |
| Tryptophan | Trpxaeiou | Trp | W |
| Tyrosine | Tyrxaeiou | Tyr | Y |
| Valine | Valxaeiou | Val | V |
| Selenocysteine | Secxaeiou | Sec | U |
| Pyrrolysine | Pylxaeiou | Pyl | O |

An alternative method for generating such distinctive names involves the utilization of algorithms like uuencoding. By employing algorithms such as uuencoding, it becomes possible to effectively generate novel names, thereby expanding the vocabulary of the system or model being employed. This approach enhances the linguistic capabilities and versatility of the system, enabling it to handle a broader range of terms and linguistic variations.

Step 2: Integrating Novel Names into the Large Language Model Vocabulary

Once the novel names have been generated and validated for their uniqueness, the subsequent step entails integrating these names into the vocabulary of the pre-trained LLM. This integration process involves adding the novel names to the existing tokens of the LLM, ensuring they become recognized and accessible for use in language generation tasks. By expanding the vocabulary of the LLM to include the newly created names, we enable the model to incorporate them seamlessly into its language generation processes, enhancing its linguistic capabilities and allowing it to generate coherent and contextually appropriate text involving these novel names.

### Step 3: Fine-Tuning a Language Model for Protein Sequence Generation

Following the vocabulary integration, the next stage involves fine-tuning the LLM using self-supervised learning techniques. To accomplish this, an extensive dataset exclusively consisting of protein sequences is constructed. It is worth noting that several protein databases, including UniProt and PDB (among others), are accessible for training purposes. In the present implementation, the model is trained using the UniProt database, which offers a rich source of protein sequence data. As illustrated in the high level process flow in FIG. 1, the LLM is trained on this dataset by utilizing the masked language modeling (MLM) methodology. The ultimate objective is to instill within the LLM a comprehensive understanding and proficiency in generating coherent protein sequences, further augmenting its capabilities in the protein domain.

In the self-supervised learning, the input protein sequence format follows a specific structure. Protein sequences are represented as strings of amino acid symbols, where each symbol corresponds to a specific amino acid residue as shown in Table 1. The sequence is typically composed of a series of these symbols, with each symbol representing an individual amino acid building block.

In protein sequence notation, each amino acid residue is denoted by a single letter symbol. For example, the amino acid alanine is represented by the symbol “A,” lysine by “K,” and so on. The protein sequence string is constructed by concatenating these symbols in the order that they appear within the protein sequence. In the self-supervised learning phase, during the training process, each single-letter symbol in the input protein sequence derived from protein databases is mapped to the corresponding new name. This mapping or translation involves replacing each amino acid symbol with the specific new name associated with it.

For example, the protein sequence “NLYIQWLKDGGPSSGRPPPS” for Trp-Cage is mapped to the corresponding sequence as follows:

Asnxaeiou-Leuxaeiou-Tyrxaeiou-Ilexaeiou-Glnxaeiou-Trpxaeiou-Leuxaeiou-Lysxaeiou-Aspxaeiou-Glyxaeiou-Glyxaeiou-Proxaeiou-Serxaeiou-Serxaeiou-Glyxaeiou-Argxaeiou-Proxaeiou-Proxaeiou-Proxaeiou-Serxaeiou

### Step 4: Training an LLM to Predict Controlled Terms for Protein Sequences

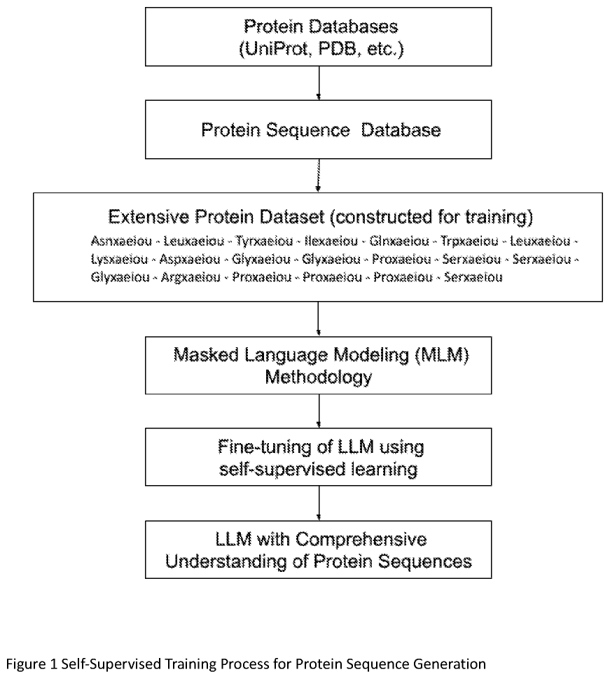

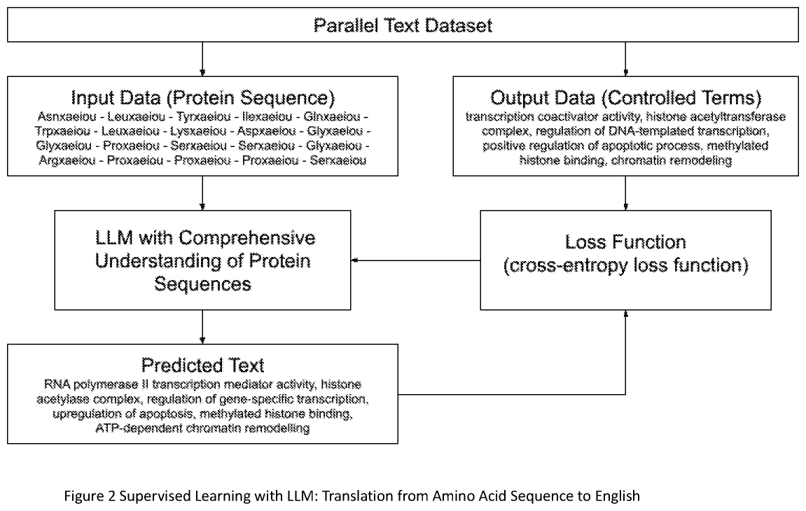

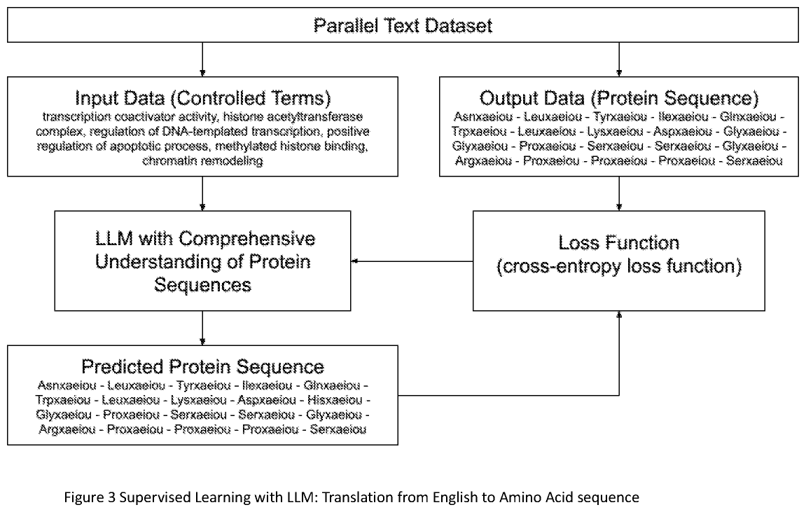

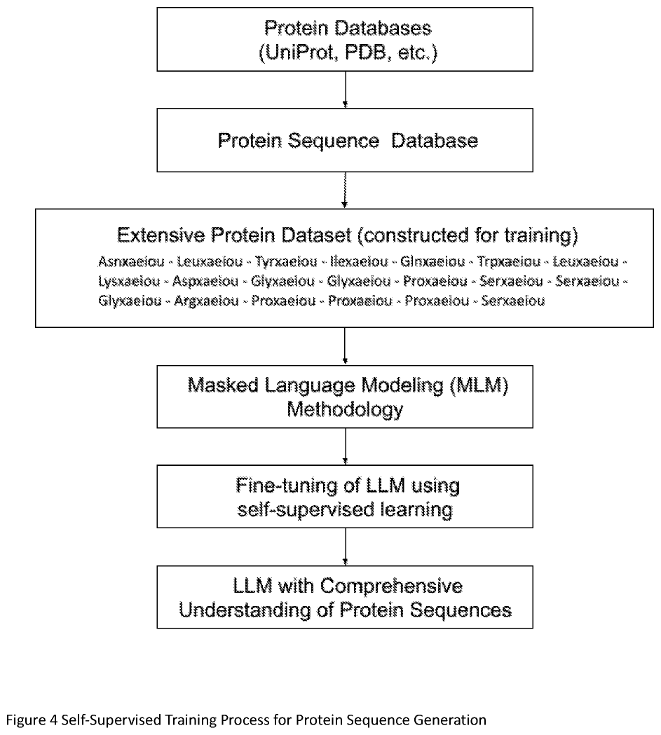

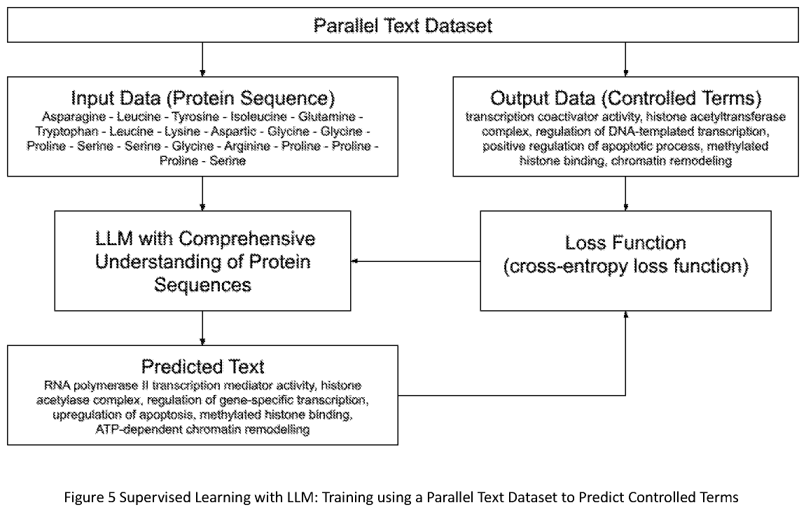

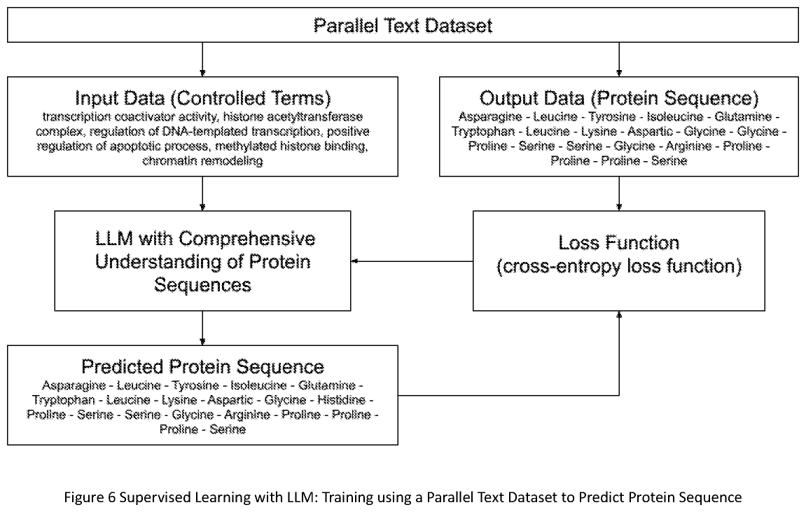

Following the completion of the self-supervised learning phase, the next step is to proceed with supervised learning. As illustrated in the high level process flow in FIG. 2, this entails curating a dataset that consists of protein sequences and their corresponding descriptions of controlled terms. These controlled terms represent the specific outcomes we want the LLM to predict when given any protein sequence. The input protein sequence format remains the same as in the self-supervised learning phase described in Step 3. For this supervised training, we again utilize the UniProt database as a source of protein sequences and controlled terms. However, It should be noted that various protein databases with similar characteristics can be utilized for this purpose, ensuring flexibility and applicability to different sources of protein sequence information.

The controlled terms associated with each protein sequence include Journals (citations/publications), Keywords, Subcellular Location, Pathways, Plasmids, Post-translational modifications, Taxonomy, Tissues, Human diseases, Extracellular domains, Interaction, and Gene Ontology. To create the training dataset, we concatenate the descriptions of the controlled terms for each protein, resulting in a label that represents the desired output. By pairing the protein sequences with their respective controlled term descriptions, the LLM is trained in a supervised manner to accurately predict these controlled terms.

Step 5: Fine-Tuning an LLM to Predict Protein Sequences from Controlled Terms

To further refine the LLM's abilities, we incorporate an additional training phase that inverses the input-output relationship of the prior training step. As demonstrated in the overarching process flow in FIG. 3, this stage focuses on the use of controlled term descriptions as the input and the associated protein sequences as the labels. The format of the protein sequence stays consistent with that described in Steps 3 and 4.

We use a publicly available protein database for sourcing protein sequences and associated controlled terms, a resource that offers a vast collection of protein data suitable for training and implementing the method.

A new dataset is assembled, which comprises controlled term descriptions as inputs and their corresponding protein sequences as labels. The descriptions include a variety of controlled terms such as Journals (citations/publications), Keywords, Subcellular Location, Pathways, Plasmids, Post-translational modifications, Taxonomy, Tissues, Human diseases, Extracellular domains, Interaction, and Gene Ontology. The goal of reversing the input-output relationship is to train the LLM to predict protein sequences based on the provided controlled term descriptions.

Utilizing the same LLM model, we carry out a supervised fine-tuning process on this dataset. The LLM learns to associate the descriptions of controlled terms with their corresponding protein sequences and fine-tunes its parameters to enhance prediction accuracy. This training allows the LLM to generate protein sequences that are contextually appropriate and align with the supplied controlled term descriptions.

### An Alternative Approach Using Pre-Existing Amino Acid Names to Fine-Tune an LLM

As an alternative, the method may commence by selecting 22 amino acid names already included in the pre-trained LLM's vocabulary. These selected names are detailed in the ‘Amino Acid Name’ column of Table 1. Utilizing these pre-existing names can aid in fine-tuning the model, capitalizing on the LLM's pre-trained understanding of these amino acids.

This alternative approach carries out steps parallel to Steps 3, 4, and 5. The controlled terms used in this iteration remain unchanged, ensuring consistency with the previous training steps. However, there is a modification in the input sequence format. Instead of using the Novel Name format, we now utilize the Amino Acid Name format as presented in Table 1. For example, the input sequence “NLYIQWLKDGGPSSGRPPPS” for Trp-Cage protein is mapped to the corresponding amino acid sequence format as follows:

Asparagine-Leucine-Tyrosine-Isoleucine-Glutamine-Tryptophan-Leucine-Lysine-Aspartic-Glycine-Glycine-Proline-Serine-Serine-Glycine-Arginine-Proline-Proline-Proline-Serine

### Step 6: Fine-Tuning the LLM to Adopt the Protein Language Model

The next stage in the process involves fine-tuning the LLM using self-supervised learning techniques. This is accomplished by constructing a large dataset that consists exclusively of protein sequences. Several protein databases, including UniProt and PDB, can be used for training purposes. In this implementation, the model is trained using the UniProt database, which offers a rich source of protein sequence data.

As shown in FIG. 4, the LLM is trained on this dataset using the masked language modeling (MLM) methodology. The ultimate goal is to instill within the LLM a comprehensive understanding and proficiency in generating coherent protein sequences. This will further augment the model's capabilities in the protein domain.

The MLM methodology involves masking out certain amino acids in a protein sequence and then asking the LLM to predict the correct amino acids. This process helps the LLM to learn the statistical relationships between different amino acids and to better understand the structure of protein sequences.

By adapting the LLM through fine-tuning on a dataset of protein sequences, we extend its capabilities, initially trained on natural language text, to encompass the protein language model as well. This enhanced model can generate more authentic and precise protein sequences, opening up a wealth of potential applications, including drug discovery and protein engineering.

### Step 7: Training an LLM to Predict Controlled Terms for Protein Sequences

Following the completion of the self-supervised learning phase, the next step is to proceed with supervised learning. As illustrated in the high level process flow in FIG. 5, this entails curating a dataset that consists of protein sequences and their corresponding descriptions of controlled terms. These controlled terms represent the specific outcomes we want the LLM to predict when given any protein sequence. The input protein sequence format remains the same as in the self-supervised learning phase described in Step 6. For this supervised training, we again utilize the UniProt database as a source of protein sequences and controlled terms. However, It should be noted that various protein databases with similar characteristics can be utilized for this purpose, ensuring flexibility and applicability to different sources of protein sequence information.

The controlled terms associated with each protein sequence include Journals (citations/publications), Keywords, Subcellular Location, Pathways, Plasmids, Post-translational modifications, Taxonomy, Tissues, Human diseases, Extracellular domains, Interaction, and Gene Ontology. To create the training dataset, we concatenate the descriptions of the controlled terms for each protein, resulting in a label that represents the desired output. By pairing the protein sequences with their respective controlled term descriptions, the LLM is trained in a supervised manner to accurately predict these controlled terms.

Step 8: Fine-Tuning an LLM to Predict Protein Sequences from Controlled Terms

To further refine the LLM's abilities, we incorporate an additional training phase that inverses the input-output relationship of the prior training step. As demonstrated in the overarching process flow in FIG. 6, this stage focuses on the use of controlled term descriptions as the input and the associated protein sequences as the labels. The format of the protein sequence stays consistent with that described in Steps 6 and 7.

A new dataset is assembled, which comprises controlled term descriptions as inputs and their corresponding protein sequences as labels. The descriptions include a variety of controlled terms such as Journals (citations/publications), Keywords, Subcellular Location, Pathways, Plasmids, Post-translational modifications, Taxonomy, Tissues, Human diseases, Extracellular domains, Interaction, and Gene Ontology. The goal of reversing the input-output relationship is to train the LLM to predict protein sequences based on the provided controlled term descriptions.

Utilizing the same LLM model, we carry out a supervised fine-tuning process on this dataset. The LLM learns to associate the descriptions of controlled terms with their corresponding protein sequences and fine-tunes its parameters to enhance prediction accuracy. This training allows the LLM to generate protein sequences that are contextually appropriate and align with the supplied controlled term descriptions.

In other words, the LLM is now able to translate natural language descriptions of protein sequences into the actual protein sequences themselves. This is a valuable capability that can be used for a variety of applications, such as drug discovery and protein engineering.

### Glossary

The following terms used herein have the following meaning:

A “Controlled Vocabulary” refers to a pre-defined and standardized list of terms or phrases used to index, categorize, and organize information in a specific domain. It ensures consistency and facilitates efficient retrieval of information. Here is a list of controlled vocabularies used in this specification:

UniProtKB: UniProtKB is a database of protein sequences and annotations. UniProtKB uses a controlled vocabulary to annotate proteins with information about their function, structure, and location.

Gene Ontology (GO): GO is a controlled vocabulary that is used to describe the functions of genes and proteins. GO terms are organized into three different ontologies: biological process, molecular function, and cellular component.

PRIDE: PRIDE is a database of protein interaction data. PRIDE uses a controlled vocabulary to annotate protein interactions with information about the type of interaction, the confidence of the interaction, and the experimental method used to identify the interaction.

Controlled Vocabulary in UniProt:

Journals (citations/publications): The controlled vocabulary of journals in UniProtKB/Swiss-Prot provides a list of journal abbreviations used in the database. The abbreviations follow the standards proposed by the International Organization for Standardization (ISO) and are typically consistent with those used by the National Library of Medicine (NLM) in PubMed. These abbreviations help standardize and identify specific journals in the context of citations and publications within UniProtKB/Swiss-Prot.

Keywords: The controlled vocabulary of keywords in UniProtKB (Swiss-Prot and TrEMBL) lists the keywords and keyword categories used for protein annotation within the knowledgebase.

Subcellular locations: The controlled vocabulary of subcellular locations and membrane topologies and orientations in UniProtKB provides standardized terms for describing the subcellular locations of proteins, including membrane topologies and orientations, in the ‘Subcellular location’ section.

Pathways: The controlled vocabulary of metabolic pathways in UniProtKB is used to annotate the ‘Pathway’ subsection of the ‘Function’ section. It defines terms related to UniPathway concepts, including pathways, sub-pathways, and enzymatic reactions (steps).

Plasmids: The controlled vocabulary of plasmids in UniProtKB lists valid values for plasmids cited in the ‘Names and origin’ section's ‘Encoded on’ subsection and the cross-references section of UniProtKB/Swiss-Prot entries.

Post-translational modifications (PTM): The controlled vocabulary of posttranslational modifications (PTM) in UniProtKB lists the PTMs used in the sequence annotation section of UniProtKB (Swiss-Prot and TrEMBL), providing information such as target amino acid, subcellular location, mass differences, taxonomic range, and corresponding keywords.

Taxonomy—Species: The controlled vocabulary of species in UniProtKB contains two sublists: real organism codes used in both UniProtKB/Swiss-Prot and UniProtKB/TrEMBL, corresponding to specific organisms, and virtual organism codes that group organisms at a certain taxonomic level, used only in UniProtKB/TrEMBL.

Strains: The controlled vocabulary of strains in UniProtKB lists frequently occurring values for the ‘Strain’ topic in the cross-references section of UniProtKB/Swiss-Prot entries.

Tissues: The controlled vocabulary of tissues in UniProtKB lists valid values and synonyms for tissues cited in the cross-references section of UniProtKB/Swiss-Prot entries.

Human diseases: The controlled vocabulary of human diseases in UniProtKB/Swiss-Prot is used for annotating human diseases. It includes disease identifiers, acronyms, descriptions, synonyms, and links to resources such as OMIM, Medical Subject Headings (MeSH), and associated UniProtKB keywords.

Extracellular domains: The document “Nomenclature of extracellular domains” provides a proposal for the nomenclature of domains found primarily in extracellular proteins of higher eukaryotes. These domains are described in the ‘Sequence annotation’ section of UniProt entries.

“Controlled terms” are the individual terms or phrases included in a controlled vocabulary. They are carefully selected and defined to represent concepts and entities within a particular domain or field.

A “Large Language Model” (LLM) is a type of artificial intelligence model designed to process and generate human-like text. LLMs, such as GPT-4, are trained on vast amounts of data to learn patterns, language structures, and contextual relationships, enabling them to generate coherent and contextually appropriate responses.

The characteristics of Large Language Models (LLMs) that distinguish them from smaller-scale language models:

Scale: LLMs have an enormous number of parameters, often ranging in the billions or even trillions. This large parameter count enables them to capture complex patterns and dependencies in language.

Pre-training on Massive Datasets: LLMs are trained on massive amounts of text data from diverse sources, such as books, articles, websites, and other publicly available text. This pre-training phase allows the models to learn the statistical regularities and patterns present in the language.

Unsupervised Learning: LLMs are trained using unsupervised learning techniques, where they learn to predict the next word in a sentence or fill in masked words given the context. This allows the models to learn general language representations without being explicitly trained on specific tasks.

Fine-Tuning for Downstream Tasks: After pre-training, LLMs can be fine-tuned on specific downstream tasks using task-specific labeled data. This fine-tuning process adapts the models to perform well on specific tasks like text classification, named entity recognition, or question answering.

Language Generation: LLMs excel at generating coherent and contextually appropriate text. They can generate human-like responses, write articles, create conversational agents, and perform language translation tasks.

Compositional Representation: LLMs learn compositional representations, meaning they capture the hierarchical and contextual relationships between words, phrases, and sentences. This allows them to understand and generate complex language structures.

Transfer Learning: LLMs exhibit strong transfer learning capabilities, meaning they can leverage the knowledge learned from pre-training on general language understanding to perform well on a wide range of downstream tasks with minimal task-specific training.

Computational Resources: Training and deploying LLMs require significant computational resources, including high-performance computing

“GPT” stands for “Generative Pre-trained Transformer.” It refers to a specific series of large-scale language models developed by OpenAI. GPT models, such as GPT-4 (the forth iteration), utilize a transformer architecture and are trained on diverse datasets to perform a range of natural language processing tasks, including text completion, translation, and question answering.

“Llama 2” is a second-generation open-source large language model (LLM) from Meta. It was released in July 2023 and is trained on a dataset of text and code that is 2 trillion tokens in size. This makes Llama 2 one of the largest and most powerful LLMs available.

“GPT-J” is a variant of the GPT (Generative Pre-trained Transformer) series, specifically referring to models based on the GPT-3 architecture that have been developed by the open-source community. GPT-J is created using publicly available code and trained on large-scale datasets by utilizing distributed computing resources. It aims to provide an accessible and open alternative to proprietary models like GPT-3, enabling researchers and developers to explore and experiment with large language models. GPT-J demonstrates similar capabilities to GPT-3 in terms of natural language understanding and generation.

“GPT-NeoX” is a 20 billion parameter autoregressive language model trained on the Pile, a massive dataset of text and code. It was released in April 2023 by EleutherAI, a research group that is dedicated to developing open-source language models.

“Self-supervised Learning” is a machine learning approach where models learn from unlabeled data without relying on explicit supervision. Instead, the models generate their own labels or use auxiliary tasks to learn useful representations of the data. In the case of language models like GPT, self-supervised learning involves training the model to predict missing or masked words within a given context.

“MLM” stands for “Masked Language Model.” It is a specific type of self-supervised learning task used in language models like GPT. In MLM, certain words or tokens in a given input text are randomly masked, and the model is trained to predict the missing masked tokens based on the context provided by the surrounding words. This task helps the model learn semantic and syntactic relationships between words and improves its understanding of language structure.

1. A method for enhancing the creativity of a Large Language Model (LLM) in protein sequence generation and predicting controlled terms from protein sequences, comprising: (a) Incorporating 22 novel names representing the 22 amino acids into the vocabulary of the LLM; (b) Conducting self-supervised learning using protein sequences from protein databases, wherein the sequences are encoded using the 22 novel names, thereby improving the LLM's comprehension and generation of coherent protein sequences involving the novel names; (c) Performing supervised learning using protein sequences as inputs and their corresponding controlled terms as outputs, thereby refining the LLM's ability to predict controlled terms based on protein sequences; and (d) Performing supervised learning using protein sequences as outputs and their corresponding controlled terms as inputs, thereby refining the LLM's ability to generate protein sequences based on controlled terms.

2. The method of claim 1, wherein the LLM is generative and pre-trained.

3. The method of claim 1, wherein the novel names are generated by a computer program or created manually.

4. The method of claim 1, wherein the dataset of protein sequences and their corresponding controlled terms is a subset of a protein database.

5. The method of claim 1, wherein the self-supervised learning is a masked language model (MLM).

6. A method for enhancing the creativity of a pre-trained Large Language Model (LLM) in protein sequence generation and predicting controlled terms from protein sequences, comprising: (a) Identifying a set of 22 amino acid names from the original vocabulary of the pre-trained LLM; (b) Conducting self-supervised learning using protein sequences from protein databases, wherein the sequences are encoded using the selected 22 amino acid names, thereby enhancing the LLM's understanding of the inherent patterns and relationships within the selected names; (c) Performing supervised learning using protein sequences with the selected names as inputs and their corresponding controlled terms as outputs, thereby reinforcing the LLM's ability to predict controlled terms based on protein sequences; and (d) Performing supervised learning using protein sequences with the selected names as outputs and their corresponding controlled terms as inputs, thereby refining the LLM's ability to generate protein sequences based on controlled terms.

7. The method of claim 6, wherein the LLM is generative and pre-trained.

8. The method of claim 6, wherein the dataset of protein sequences and their corresponding controlled terms is a subset of a protein database.

9. The method of claim 6, wherein the self-supervised learning is a masked language model (MLM).
